(19) United States
(12) Patent Application Publication
(10) Pub. No.: US 2018/0100821 A1
(43) Pub. Date: Apr. 12, 2018

(54) Method for testing at least one disinfectant

(71) Applicant: Brandes Innovation, Schortens (DE)

(72) Inventors: Ronald BRANDES, Schortens (DE); Joachim WILLMS, Oldenburg (DE)

(21) Appl. No.: 15/726,972

(22) Filed: Oct. 6, 2017

(30) Foreign Application Priority Data
Oct. 7, 2016 (DE) 10 2016 119 096.9

(51) Int. Cl. G01N 27/06; G01N 9/36; G01N 27/30

(52) U.S. Cl. CPC G01N 27/06; G01N 27/302; G01N 9/36

(57) ABSTRACT

In a method for testing the disinfecting activity of at least one disinfectant it is provided that at least one sample of the disinfectant is measured with a sensor in regard to a change in at least one physical and/or chemical property of the disinfectant.

The invention relates to a method for testing the disinfecting activity of at least one disinfectant.

Disinfectants are used for killing microbes in many environments. They are regularly used in hospitals, children's homes or old-age homes, in which large numbers of pathogenic organisms may be present. Disinfectants are, for example, kept in containers from which disinfectants may be taken for spraying the hands, for spraying cloths or the like.

One problem is that pressure situations frequently occur in such hospitals and homes, relating to high patient or resident populations, low staffing, or large numbers of trainees or community service workers. All this can mean that a disinfectant is held in a specific container for weeks or even months without attention being paid to its expiration date. A disinfectant of this type can lose its disinfecting activity, and indeed it may even become contaminated with microbes. Then a disinfectant would become a source of microbes.

The invention is based on the task of specifying a method of the initially mentioned class with which the disinfecting activity of a disinfectant can be reliably tested.

This goal is accomplished according to the invention in that at least one sample of the disinfectant is measured with a sensor in regard to a change in at least one physical and/or chemical property of the disinfectant.

The method according to the invention suggests active testing of disinfectants during their use, for example, in hospitals. According to the method, at least one sample of the disinfectant can be taken, or alternatively the method can be performed directly in the specific disinfectant reservoir being tested. The sample obtained or the disinfectant is measured with a sensor. This can, in an automated manner, measure a change in a physical and/or a chemical property of the disinfectant and thus determine a measurement result and supply an evaluation via suitable interfaces or transmission means. The sensor measures the physical and/or chemical property of the disinfectant. If the disinfectant still has high disinfecting efficacy, its physical and/or chemical properties will be in accordance of the specifications of the disinfectant manufacturer. If on the other hand the disinfecting property of the disinfectant is bound by a higher concentration of bacteria in the disinfectant, physical and/or chemical properties change, and this will be determined with the sensor.

For example, a long utilization time of a disinfectant changes the conductivity of a disinfectant. This change can be determined with a conductivity sensor. Alternatively the sensor can be designed as a photosensor, specifically when a long service time of a disinfectant results in a change in its turbidity.

Alternatively the sensor can be designed as a pH meter or a density sensor.

1. A method for testing the disinfecting activity of at least one disinfectant,
wherein
at least one sample of the disinfectant is measured with a sensor in regard to a change in at least one physical and/or chemical property of the disinfectant.

2. The method according to claim 1, wherein a conductivity sensor is used as the sensor.

3. The method according to claim 1, wherein a photosensor is used as the sensor.

4. The method according to claim 1, wherein a pH meter is used as the sensor.

5. The method according to claim 1, wherein a density sensor is used as the sensor.
